(19) United States
(12) Patent Application Publication
(10) Pub. No.: US 2010/0211182 A1
(43) Pub. Date: Aug. 19, 2010

(54) Thermally Sprayed Surface Layer As Well As An Orthopedic Implant

(76) Inventor: Harald Zimmermann, Wohlen (CH)

Correspondence Address: ROBERT S. GREEN, SULZER METCO (US), INC., 1101 PROSPECT AVENUE, WESTBURY, NY, 11590, US

(21) Appl. No.: 12/635,262

(22) Filed: Dec. 10, 2009

(30) Foreign Application Priority Data
Dec. 16, 2008 (EP) 08171821.5

(51) Int. Cl. A61F 2/28

(52) U.S. Cl. 623/23.53

(57) ABSTRACT

The invention relates to a thermally sprayed surface layer of titanium (21) on a non-metal substrate (3) of an orthopedic implant (1). In accordance with the invention, the thermally sprayed surface layer (2) includes an X-ray sensitive admixture with respect to the titanium (21), wherein the X-ray sensitive admixture includes a biocompatible indicator metal (4) between 0.01 at % and 20 at %, wherein the atomic weight of the biocompatible indicator metal (4) is larger than the atomic weight of titanium (21). Furthermore, the invention relates to an orthopedic implant (1) with a thermally sprayed surface layer (2).

The invention relates to a thermally sprayed surface layer of titanium on a non-metal substrate of an orthopedic implant as well as to an orthopedic implant in accordance with the preamble of the respective independent claim.

Today orthopedic implants are frequently manufactured from titanium alloys, stainless steel or CoCrMo alloy. For the better adhesion of the bone and for the acceleration of the healing process, the implants are typically provided with a porous titanium layer and/or a biocompatible hydroxyl apatite layer. The previously mentioned metals and metal alloys can, for example, be excellently localized in an X-ray picture so that the surgeon can directly judge the success of his work with reference to an X-ray picture.

In recent years non-metallic materials have become more and more important as a new substrate material for orthopedic implants. A very important material which is very biocompatible and is therefore successfully used in a wide variety of all types of medical applications, is the very well known polyether ether keton (PEEK), with a corresponding substrate of PEEK being able to be strengthened with carbons fibre in particular for orthopedic applications. Naturally, for special applications other plastic materials can also be used. In principle substrates are also possible which are completely made up of a carbon fibre material or from other non-metal materials.

The disadvantage of these materials which are very innovative per se is that they cannot be identified in an X-ray picture, as they practically do not contain any heavy metal atoms. Even when the substrate is provided with a titanium coating, for example by means of a thin thermal titanium layer or with a hydroxyl apatite layer, the implants remain in visible in the X-ray picture to a large extent. The comparatively light titanium atoms namely have a comparatively small interaction cross-section for X-ray light so that a thinly sprayed thermal spray layer can practically not be seen on an X-ray picture.

So that the surgeon can nevertheless localize an implanted implant on an X-ray picture, it is known to likewise provide the normally injection moulded implants and/or substrates with the likewise biocompatible tantalum wires. Since tantalum has an atomic weight which is approximately four times that of titanium, tantalum can be seen significantly better on an X-ray picture, even in relatively small amounts.

It is obvious that the preparation of the implants with tantalum wires is very complex, complicated and therefore cost intensive affair. Furthermore, the implants prepared in such a way are also only coarsely localizable on the X-ray picture, since the spatial resolution is ultimately restricted by the mutual spacing of neighbouring tantalum wires. To increase the spatial resolution the density of the tantalum wires has to be increased in the substrate which only increases effort and/or cost even more.

In principle the implants could also be coated with a thermal tantalum coating by thermal spraying. However, this is not possible since not only the biocompatibility of the material per se plays a role. But also the surface area structure has a significant influence on the adhesion of the bone to the surface of the implant and on the healing process. A more or less pure tantalum surface is therefore to be avoided.

A titanium surface is rather wanted which not only has an excellent biocompatibility from a chemical point of view, but rather also has a specific surface area structure which accelerate the adhesion at the bones and significantly increase the healing process. With orthopedic implants which have a non-metal substrate there has previously been no suitable alternative to the use of tantalum wires for the localization of the implant in the human or animal body.

It is therefore the object of the invention to provide a thermally sprayed surface layer of titanium on a non-metal substrate which has all the known excellent biocompatible properties of a conventional titanium coating and can simultaneously be identified on an X-ray picture adequately enough with a high spatial resolution for the localization of the substrate in the human body or the animal body.

A further object of the invention is to provide a corresponding orthopedic implant having a thermally sprayed surface layer of titanium.

The subject matters satisfying these objects of the invention are characterized by the features of the respective independent claims.

The associated dependent claims relate to particularly advantageous embodiments of the invention.

The invention thus relates to a thermally sprayed surface layer of titanium on a non-metal substrate of an orthopedic implant. In accordance with the invention, the thermally sprayed surface layer includes an X-ray sensitive admixture with respect to titanium with the X-ray sensitive admixture including a biocompatible indicator metal between 0.01 at % and 20 at % with the atomic weight of the biocompatible indicator metal being larger than the atomic weight of titanium.

The present invention therefore simultaneously uses the excellent biocompatible properties of the titanium coating or layer and the X-ray sensitive properties of a biocompatible indicator metal which is provided in a relatively small concentration as an admixture to the surface layer of titanium which is thermally sprayed onto the non-metal substrate by means of a thermal spraying process. Since the X-ray sensitive indicator metal is provided only as an admixture in a relatively small concentration to the titanium coating, the actual structure of the titanium coating remains essentially unchanged so that the layer built-up in this manner can not only be excellently localized in an X-ray picture due to the indicator metal. At the same time the surface coating in accordance with the invention has all the excellent biocompatible properties of the known conventional titanium surface layers so that in particular not only an optimal adhesion of the bone to the surface layer in accordance with the invention but also the acceleration of the healing process is guaranteed without limitation by the titanium structure.

The titanium powder used for thermal coating can for this purpose simply be so to say contaminated with a relatively small concentration of an indicator metal. In this respect, an important example of an indicator metal is tantalum which has an atomic weight which is approximately four times higher than that of titanium and can therefore also be localized excellently in an X-ray picture even in small concentrations.

A further substantial advantage is that no alloy has to be formed between the titanium and the indicator metal. The titanium powder and a powder of indicator metal are rather preferably simply thermally sprayed at the same time, with commercially available spraying powders simply being able to be used which need not be further conditioned prior to the thermal spraying. In particular it is not necessary to make an alloy of the two powders or to subject these to a special mixing procedure. As long as the powders otherwise fulfil all the necessary medical specifications, they can be thermally sprayed onto the non-metal substrate of the implant without any further conditioning.

Thus for the first time by the present invention one can dispense with the cost-intensive and demanding technique of the preparation of the implant with tantalum wires when using non-metal substrates.

It has been found that a proportion of indicator metal in relation to the total amount of titanium in the surface layer should contain between 0.01 atomic percent (at %) and 20 atomic percent (at %) so that the structural properties of the titanium coating are not distorted by too large an admixture of tantalum, but at the same time the indicator metal still being visible in sufficient clarity and spatial resolution in the X-ray picture. If significantly less tantalum than 0.01 at % is contained in the titanium layer then the localizability of the implant on an X-ray picture becomes unusably bad. If significantly more than 20 at % of tantalum is admixed to the titanium surface, the structure of the titanium layer increasingly changes such that an adhesion of the bone to the implant is no longer promoted sufficiently and also the healing process is no longer supported as wanted.

Preferably, the proportion of the indicator metal actually only lies between 0.1 at % and 10 at % and depending on the indicator metal used between 0.5 at % and 5 at %.

In this respect, the indicator metal is a metal from the group of metals including tantalum, gold, platinum and hafnium. All the aforesaid metals have the required biocompatible properties and at the same time have a sufficiently large atomic weight so that they are perfectly suitable as indicator metals in the sense of the present invention.

Even when an alloy of the indicator metal with the titanium of the surface layer is not necessary the indicator metal can naturally still be partially or totally alloyed with the titanium.

In this respect, the indicator metal itself can be an alloy of two or more metals from the group of metals including tantalum, gold, platinum and hafnium.

In an embodiment particularly important for practice, the thermally sprayed surface layer only includes titanium and the indicator metal apart from any medically and technically insignificant contaminants. In this respect, it is guaranteed that the surface layer in accordance with the invention contains no medically critical impurities.

In a further important embodiment, the thermally sprayed surface layer is formed as a titanium matrix having regions of embedded indicator metal. This means that the structure of the sprayed surface layer is such that small regions, preferably locally isolated regions consisting of the indicator metal is embedded in a layer of titanium. For example, this can be achieved when the thermal spraying process is conducted such that the indicator metal is essentially not molten on the thermal spraying process, is at most perhaps starting to melt on the surface of the powder grains and are therefore included as isolated regions in the titanium matrix.

Furthermore, the invention relates to an orthopedic implant including a non-metal substrate with a thermally sprayed surface layer of titanium. In accordance with the invention, the thermally sprayed surface layer includes an X-ray sensitive admixture with respect to the titanium with the X-ray sensitive admixture including a biocompatible indicator metal between 0.01 at % and 20 at % with the atomic weight of the biocompatible indicator metal being larger than the atomic weight of titanium.

Preferably, the proportion of the indicator metal even lies between only 0.1 at % and 10 at % and depending on the indicator metal used only lies between 0.5 at % and 5 at % with the indicator metal being a metal from the group of the metals including tantalum, gold, platinum and hafnium, with the indicator metal also being able to be an alloy of two or more metals from the group of metals including tantalum, gold, platinum and hafnium.

Particularly preferably, the thermally sprayed surface layer only includes titanium and the indicator metal apart from any contaminants, with the thermally sprayed surface layer being particularly advantageously, but not necessaryily, formed as a titanium matrix with regions of embedded indicator metal.

In this respect, the orthopedic implant of the present invention preferably includes a substrate of a biocompatible plastic, in particular of PEEK and/or of a carbon fibre material, in particular of a PEEK carbon fibre compound material.

Advantageously, a biocompatible hydroxyl apatite layer can be provided on the thermally sprayed surface layer which, in addition to the porous titanium, contributes to the acceleration of the healing process and likwise positively supports the adhesion to the bone.

In particular, an orthopedic implant of the present invention can, for example, be a shoulder implant, an elbow implant, a spinal column implant, a hip joint implant, a knee implant, a finger implant or any other orthopedic implant.

The invention will be explained in more detail in the following with reference to the schematic drawing. There are shown:

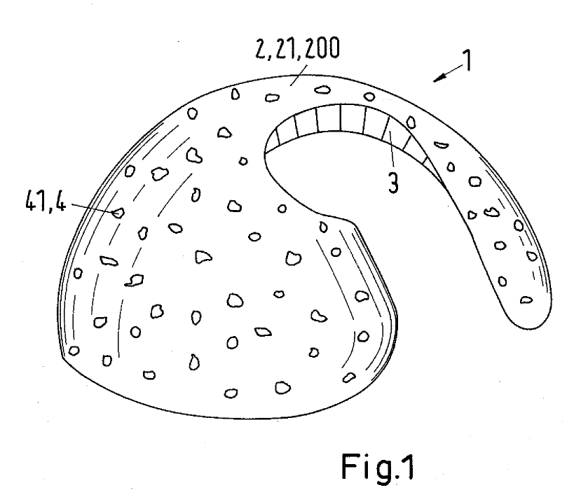

FIG. 1 an acetabulum with a surface layer in accordance with the invention;

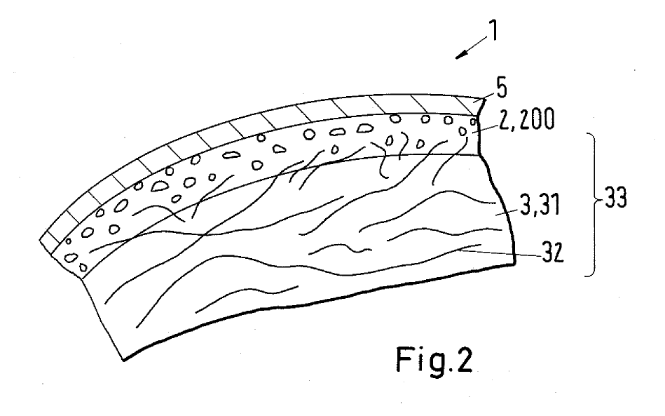

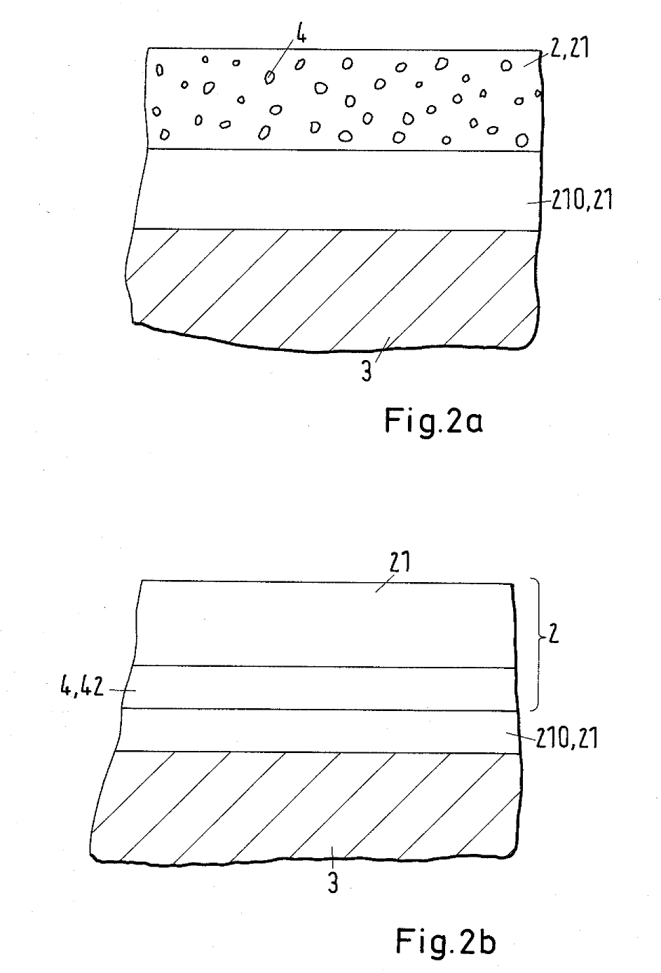

FIG. 2 a section of the surface layer in accordance with FIG. 1;

FIG. 2a an implant with a surface layer and an adhesion layer;

FIG. 2b an implant in accordance with FIG. 2 with an indicator layer;

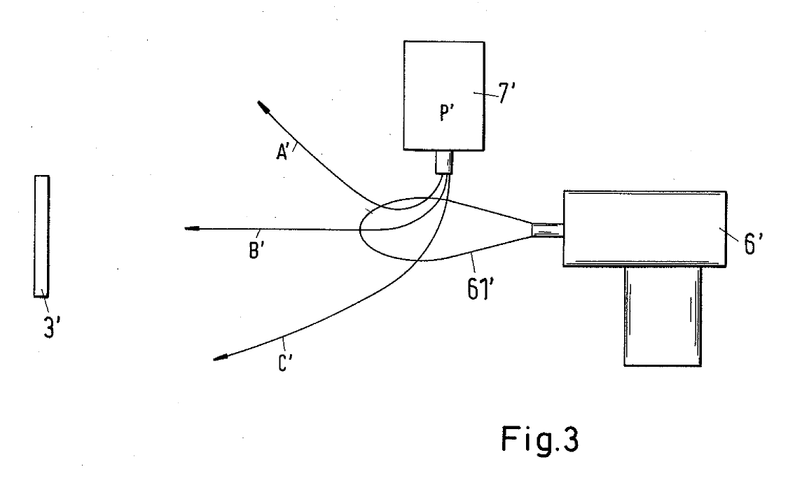

FIG. 3 a known thermal spraying process with different spraying materials;

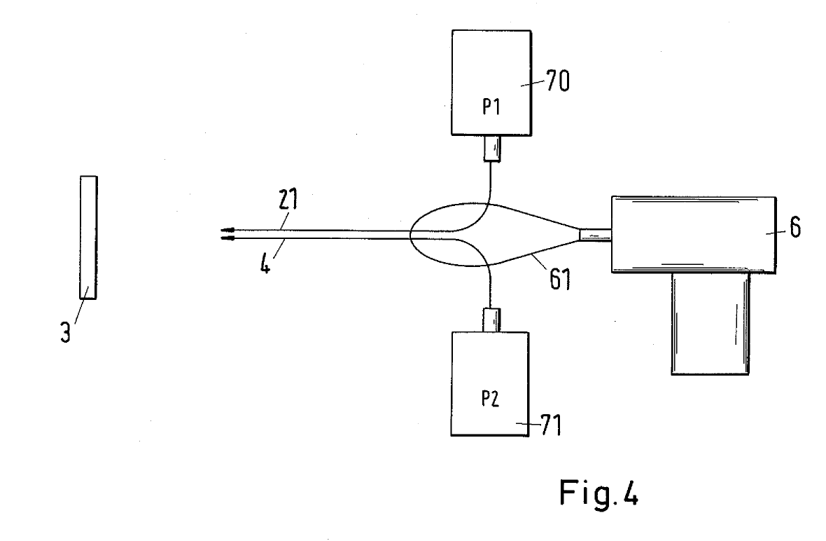

FIG. 4 a spraying process with different spraying materials for the production of a surface layer in accordance with the invention.

FIG. 1 schematically illustrates a perspective view of an acetabulum with a surface layer in accordance with the invention. FIG. 2 shows a section of the surface layer in accordance with FIG. 1.

The acetabulum 1 in accordance with FIG. 1 and/or FIG. 2 includes a non-metal substrate 3 with a thermally sprayed surface layer 2 of titanium 21. The thermally sprayed surface layer 2 of FIGS. 1 and 2 includes an X-ray sensitive admixture in relation to the titanium 21 of approximately 2 at % or 5 at % respectively of a biocompatible indicator metal 4 with the indicator metal 4 being tantalum 4 in the present example whose atomic weight is approximately four times larger than the atom weight of titanium so that the acetabulum 1 can be localized very well by the surgeon in an X-ray picture.

In this respect, the thermally sprayed surface layer 2 consists only of titanium 21 and tantalum 4 apart from any technically and medically irrelevant contaminants.

As can clearly be seen, the thermally sprayed surface layer 2 is formed as a titanium matrix 200 with regions 41 of embedded tantalum 4.

In the examples of FIG. 1 and FIG. 2 the substrate 3 is made up of a PEEK carbon fibre compound material 33, i.e. the substrate 3 of the acetabulum 1 consists of PEEK into which carbon fibres 32 are embedded in a manner known per se for the strengthening of the PEEK 31.

As can be easily be recognized, the carbon fibres 32 partially protrude out of the surface of the substrate 3. The thermal surface layer 2 thus simultaneously serves to bind the protruding carbon fibres 32 so that the protruding carbon fibres 32 can not enter into damaging interactions with the tissues of a human or animal body into which the acetabulum 1 is implanted.

In addition, a biocompatible hydroxyl apatite layer 5 is provided on the thermally sprayed surface layer 2 which additionally positively influences the adhesion of the bone to the acetabulum 1 and also the healing in general.

FIG. 2a and FIG. 2b show by way of example an implant 1 with further variants of thermally sprayed surface layers 2 in accordance with the present invention.

FIG. 2a shows a schematic section of an orthopedic implant 1 in which an additional adhesion layer 210 is provided between the surface layer 2 in accordance with the invention and the substrate 3, with in the present case the adhesion layer being made up of titanium 21. The adhesion of the surface layer 2 to the substrate 3 of the implant 1 is significantly improved by the provision of the adhesion layer 210 which adheres better on the substrate 3 than the surface layer 2 in accordance with the invention and at the same time also adheres excellently to the surface layer 2.

FIG. 2b schematically shows a further specific embodiment of a surface layer 2 in accordance with the invention which in the present case is formed by a two layer system of an outer surface layer of pure titanium 21 and of an indicator layer 42 lying beneath it essentially only includes the indicator metal 4. An adhesion layer 210 of titanium 21 is again provided between the indicator layer 42 and the substrate 3 since the adhesion layer 210 of titanium 21 adheres better to the substrate 3 than the indicator layer 42 and at the same time very well to the indicator layer 42. The admixture of the biocompatible indicator metal is therefore achieved by the formation a two layer system of the indicator layer 42 and of the outer layer of titanium 21.

In this respect, an adhesion layer 210 can be provided even when, for example, the melting point of the indicator metal 4 is comparatively high. It is then possible for the coating of the substrate 3 with an indicator metal 4, if the flame energy of the thermal spraying process would have to be chosen so high that the substrate 3 made of a temperature sensitive plastic, for example, would be damaged during the spraying process. Advantageously in this case, initially an adhesion layer 210 is applied to the substrate 3 which has a lower melting point than the indicator metal 4. The surface of the substrate 3 is thereby protected by the adhesion layer 210 onto which, for example, an indicator layer 42 of the higher melting indicator metal 4 can then be applied.

The person of ordinary skill understands that other combinations of layers up to multi-layered systems can naturally also advantageously be formed and that, for example, materials other than titanium can also be advantageously used for the adhesive layer.

In this respect for the production of a surface layer 2 in accordance with the invention, however, a particular thermal spraying process must be used. The reason for this is that two different commercially available powders are simply mixed as the spraying material.

Depending on the specific demands on the substrate, it is possible that the mass of the individual powder particles of the two spraying powders are significantly different. As a rule this will be the case when the powder particles of the titanium spray powder and of the spray powder of the indicator metal, i.e. for example tantalum, have approximately the same size and/or the same size distribution.

For many applications, this is of particular advantage together with a similar morphology of the two spraying powders since otherwise a de-mixing can easily be achieved so that the regions of the indicator metal are distributed very irregularly in the metallic matrix of titanium. This naturally has to be prevented. This means that, for example, the powder grains of both powders have a spherical shape or both have a rectangular shape or both have an edged broken structure. If both powder kinds additionally have powder grains of comparable sizes a de-mixing of the two powders is rather not to be expected during the thermal spraying.

However, as the indicator metal has to have a higher atomic weight than titanium, the powder grains of the indicator metal inevitably have a higher mass than the titanium grains if the two different powder grains have approximately the same size.

This can lead to severe complications on spraying as is impressively demonstrated in accordance with FIG. 3. FIG. 3 shows a spraying process known from the prior art which is carried out with three different types of powder grains A′, B′ and C′ which have approximately the same size, but significantly different masses.

For a significant differentiation of the invention from the prior art, the features known from the prior art are provided with a dash in FIG. 3 whereby the features of the present invention have no dash. So also not the features in accordance with FIG. 4 which show a method for the production of a thermal surface layer of titanium in accordance with the invention.

In the spraying process illustrated in FIG. 3, three different spraying powders A′, B′ and C′ are supplied to the plasma flame 61′ of the spraying pistol 6′ at the same time via the powder injector 7′. In this respect, the powder injector 7′ is operated at a working pressure P′ at which the powder particles A′, B′ and C′ are injected into the plasma flame 61′.

In this respect the powder grains A′ are lighter than the powder grains B′ and they are in turn lighter than the powder grains C′.

This leads to the fact that the working pressure P′ at which the powder particles A′, B′ and C′ are injected into the plasma flame 61′ can only be adapted and can then only be ideal for the particle mass of a particle type, in the present case for the mass of the particles B′.

For the lighter particles A′ the working pressure P′ is too small. They are quasi reflected at the plasma flame 61′ and can at least only be ingested in an insufficient amount to the plasma flame 61′. The mass of the particles B′ is in turn too large for the working pressure P′ so that the particles B′ are at least partially swung through the plasma flame 61′.

The negative result is clear: in the thermal spraying layer which should be sprayed onto the substrate 3′, to large a proportion of material of the type B′ is present, whereas too little material of the type A′ and C′ will be present in the thermal spraying layer.

The present invention avoids this problem in that, in accordance with FIG. 4 for the production of a surface layer 2 in accordance with the invention, for example, two powder injectors 70 and 71 are used. The powder injector 70 in this respect provides titanium powder 21 and the powder injector 71 provides the indicator metal 4 as is indicated by the corresponding arrows in FIG. 4. In this respect, two powder injectors 70, 71 can naturally also be accommodated in a single injector module.

The working pressures at which both powder injectors 70 and 71 are used are different in this respect and are ideally adjusted for the different mass of the titanium powder 21 and of the indicator metal powder 4 so that both powder types 21, 4 are ideally injected into the plasma flame 61 of the plasma spraying pistol 6 so that a thermally sprayed surface layer in accordance with the invention can be manufactured on the substrate 3 in a high quality and of the desired composition.

In a different variant, a powder mixture of the titanium and the indicator metal is produced, with the grain size distribution and the morphology of titanium and/or of the indicator metal being matched such that the powder can be supplied to the plasma flame via a single powder injector at a uniform working pressure. This is preferably achieved in that, for example, one mixes larger titanium powder grains with smaller indicator powder grains, with the size and/or the size distribution of the two particle types being chosen such that the mass and/or the mass distribution of both particle types is/are matched to one another such that both can be introduced ideally into the plasma flame and by means of the same powder injector at the same working pressure. In a preferred embodiment, the size and/or the size distribution of the two particle types is chosen such that the mass and/or the mass distribution of both particle types is approximately the same.

As previously mentioned, in a preferred embodiment the small indicator metal grains, for example, the small tantalum grains do not necessarily have to melt in the thermal spraying process, but they can be enclosed into the forming titanium matrix. In this respect, preferably a stirring device is provided at a powder conveyor which conveys the spraying powder to the powder injector so that the spraying powder is constantly well mixed prior to the powder mixture of titanium and of indicator metal being injected into the plasma flame.

In this respect, it is to be understood that the invention is not restricted to the described embodiments and in particular the embodiments in accordance with the invention described in the framework of this invention can naturally also be combined in every suitable way.

1. A thermally sprayed surface layer of titanium (21) on a non-metal substrate (3) of an orthopedic implant (1), characterized in that the thermally sprayed surface layer includes an X-ray sensitive admixture with respect to the titanium (21), wherein the X-ray sensitive admixture includes a biocompatible indicator metal (4) between 0.01 at % and 20 at %, wherein the atomic weight of the biocompatible indicator metal (4) is larger than the atomic weight of titanium (21).

2. A thermally sprayed surface layer in accordance with claim 1, wherein the proportion of the indicator metal (4) lies between 0.5 at % and 5 at %.

3. A thermally sprayed surface layer in accordance with claim 1, wherein the indicator metal (4) is a metal from the group including tantalum, gold, platinum and hafnium.

4. A thermally sprayed surface layer in accordance with claim 3, wherein the indicator metal (4) is an alloy of two metals from the group including tantalum, gold, platinum and hafnium.

5. A thermally sprayed surface layer in accordance with claim 1, wherein the thermally sprayed surface layer includes, apart from any contaminants, titanium (21) and the indicator metal (4).

6. A thermally sprayed surface layer in accordance with claim 1, wherein the thermally sprayed surface layer is formed as a titanium matrix (200) having regions (41) of embedded indicator metal (4).

7. An orthopedic implant including a non-metal substrate (3) with a thermally sprayed surface layer (2) of titanium (21), characterized in that the thermally sprayed surface layer (2) includes an x-ray sensitive admixture with respect to the titanium (21), wherein the x-ray sensitive admixture includes a biocompatible indicator metal (4) between 0.01 at % and 20 at %, wherein the atomic weight of the biocompatible indicator metal (4) is larger than the atomic weight of titanium (21).

8. An orthopedic implant in accordance with claim 7, wherein the proportion of the indicator metal (4) lies between 0.5 at % and 5 at %.

9. An orthopedic implant in accordance with claim 7, wherein the indicator metal (4) is a metal from the group including tantalum, gold, platinum and hafnium.

10. An orthopedic implant in accordance with claim 9, wherein the indicator metal (4) is an alloy of two metals from the group including tantalum, gold, platinum and hafnium.

11. An orthopedic implant in accordance with claim 7, wherein the thermally sprayed layer (2) includes, apart from any contaminants, titanium (21) and the indicator metal (4)

12. An orthopedic implant in accordance with claim 7, wherein the thermally sprayed surface layer (2) is formed as a titanium matrix (200) having regions (41) of embedded indicator metal (4).

13. An orthopedic implant in accordance with claim 7, wherein the substrate (3) includes a biocompatible plastic, in particular PEEK (31) and/or a carbon fiber material (32), in particular a PEEK carbon fiber composite material (33).

14. An orthopedic implant in accordance with claim 7, wherein a biocompatible hydroxylapatite layer (5) is provided on the thermally sprayed surface layer (2).

15. An orthopedic implant in accordance with claim 7, wherein the orthopedic implant is a shoulder implant, an elbow implant, a spinal column implant, a hip joint implant, a knee implant, a finger implant or any other orthopedic implant.
